(19) United States
(12) Patent Application Publication
(10) Pub. No.: US 2015/0199480 A1
(43) Pub. Date: Jul. 16, 2015

(54) CONTROLLING HEALTH SCREENING VIA ENTERTAINMENT EXPERIENCES

(71) Applicant: Microsoft Corporation, Redmond, WA (US)

(72) Inventors: Paul Henry Dietz, Redmond, WA (US); Desney S. Tan, Kirkland, WA (US); Timothy Scott Saponas, Woodinville, WA (US); Daniel Scott Morris, Bellevue, WA (US); Andrew David Wilson, Seattle, WA (US)

(21) Appl. No.: 14/567,002

(22) Filed: Dec. 11, 2014

Related U.S. Application Data
(60) Provisional application No. 61/927,778, filed on Jan. 15, 2014.

(51) Int. Cl. G06F 19/00; A61B 5/021; A61B 5/055; A61B 8/08; A61B 5/1455; A61B 5/00; A61B 5/0476; A61B 5/0488; A61B 5/0496; A61B 5/0402; A61B 6/00

(52) U.S. Cl. CPC G06F 19/322; A61B 5/0402; A61B 5/021; A61B 5/055; A61B 8/0883; A61B 6/503; A61B 5/4884; A61B 5/0476; A61B 5/0488; A61B 5/0496; A61B 5/14551; A61B 5/4806

(57) ABSTRACT

Various technologies described herein pertain to controlling performance of a health assessment of a user in an entertainment venue. Data in a health record of the user is accessed, where the health record is retained in computer-readable storage. The user is located at the entertainment venue, and the entertainment venue includes an attraction. A health parameter of the user to be measured as part of the health assessment performed in the entertainment venue is selected based on the data in the health record of the user. Further, an interaction between the user and the attraction of the entertainment venue is controlled based on the health parameter to be measured. Data indicative of the health parameter of the user is computed based on a signal output by a sensor. The signal is output by the sensor during the interaction between the user and the attraction of the entertainment venue.

### CROSS-REFERENCE TO RELATED APPLICATION

This application claims priority to U.S. Provisional Patent Application No. 61/927,778, filed on Jan. 15, 2014, and entitled “PROVIDING HEALTH SCREENING VIA ENTERTAINMENT EXPERIENCES”, the entirety of which is incorporated herein by reference.

### BACKGROUND

Regular health screening can be critical for early detection of treatable medical conditions. Unfortunately, many people conventionally fail to participate in regular health screenings as frequently as they should (e.g., people may go a number of years between physicals, etc.). A factor that commonly leads to infrequent health screening for a portion of the population is a lack of insurance coverage. Another factor that typically causes a portion of the population to fail to participate in regular health screening is that a health screen is oftentimes an unpleasant activity.

Various aspects of conventional health screens can be unpleasant. For example, appointments for health screens typically are scheduled well in advance; thus, a patient who fails to plan ahead may be unable to have a health screen conducted. Moreover, when finally going to a health screen appointment, a patient commonly waits in a waiting room of a medical facility filled with people that may have various illnesses. Accordingly, a person who is healthy prior to the health screen may be exposed to various contagious illnesses in the waiting room, and thus, may become ill as a result of going to the medical facility for having the health screen performed. Further, conventional health screens are oftentimes conducted in sterile medical environments that patients typically encounter when sick. As a result, people may react poorly to this association, showing increased heart rate, higher blood pressure, etc., thereby making measurements obtained during the health screen less valid.

There have been various conventional attempts that try to make the process of health screening more pleasant. For instance, a medical facility for conducting a health screen may be decorated in a homelike manner to try to place the patients at ease. Further, a health screen can be performed as part of a wellness fair, where the health screen is conducted offsite from the medical facility, yet still in a clinical manner. Other common techniques include concierge medicine, providing patients (e.g., children) with items such as lollipops or stickers, and so forth.

### SUMMARY

Described herein are various technologies that pertain to controlling performance of a health assessment of a user in an entertainment venue. Data in a health record of the user can be accessed, where the health record is retained in computer-readable storage. The user can be located at the entertainment venue, and the entertainment venue includes an attraction (or a plurality of attractions). A health parameter of the user can be selected to be measured as part of the health assessment performed in the entertainment venue. The health parameter can be selected based on the data in the health record of the user. Further, an interaction between the user and the attraction of the entertainment venue can be controlled based on the health parameter to be measured. Moreover, data indicative of the health parameter of the user can be computed based on a signal output by a sensor. The signal can be output by the sensor during the interaction between the user and the attraction of the entertainment venue.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF THE DRAWINGS

FIG. 1 illustrates a functional block diagram of an exemplary system that provides health screening via entertainment experiences.

FIG. 2 illustrates a functional block diagram of an exemplary system that controls performance of a health assessment of a user in an entertainment venue.

FIG. 3 illustrates a functional block diagram of another exemplary system that controls performance of a health assessment of the user in the entertainment venue.

FIG. 4 illustrates a functional block diagram of an exemplary system that tailors an experience of the user when interacting with an attraction in the entertainment venue.

FIG. 5 illustrates a functional block diagram of an exemplary system that distributes data from a health record of the user to a computing device.

FIG. 6 illustrates a functional block diagram of an exemplary system that controls personalization of treatment based on data collected from the user at the entertainment venue.

FIG. 7 illustrates a functional block diagram of an exemplary system that controls personalization of an educational experience for the user based on a detected condition.

FIG. 8 illustrates a functional block diagram of an exemplary attraction of the entertainment venue.

FIG. 9 is a flow diagram that illustrates an exemplary methodology of controlling performance of a health assessment of a user in an entertainment venue.

FIG. 10 is a flow diagram that illustrates an exemplary methodology of controlling personalization of treatment of a user detected to have a health condition in an entertainment venue.

FIG. 11 illustrates an exemplary computing device.

FIG. 12 illustrates an exemplary computing system.

### DETAILED DESCRIPTION

Various technologies pertaining to providing health screening via entertainment experiences are now described with reference to the drawings, wherein like reference numerals are used to refer to like elements throughout. In the following description, for purposes of explanation, numerous specific details are set forth in order to provide a thorough understanding of one or more aspects. It may be evident, however, that such aspect(s) may be practiced without these specific details. In other instances, well-known structures and devices are shown in block diagram form in order to facilitate describing one or more aspects. Further, it is to be understood that functionality that is described as being carried out by certain system components may be performed by multiple components. Similarly, for instance, a component may be configured to perform functionality that is described as being carried out by multiple components.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

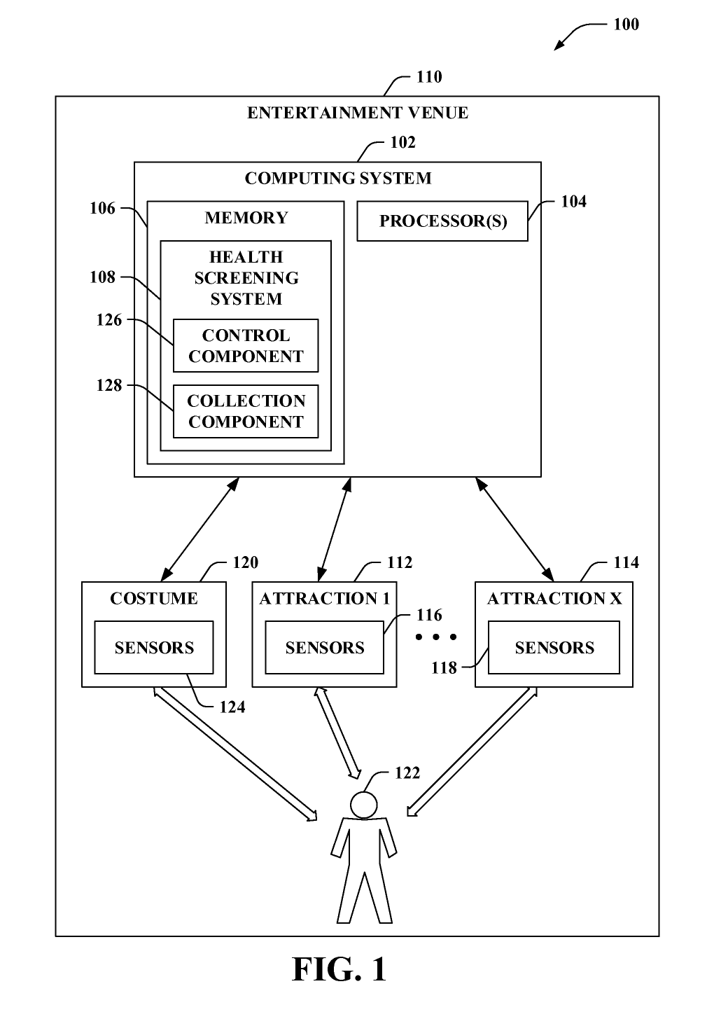

Referring now to the drawings, FIG. 1 illustrates a system 100 that provides health screening via entertainment experiences. The system 100 includes a computing system 102. The computing system 102 includes one or more processors 104 and memory 106. The processor(s) 104 are configured to execute instructions loaded into the memory 106 (e.g., one or more systems loaded into the memory 106 are executable by the processor(s) 104, one or more components loaded into the memory 106 are executable by the processor(s) 104, etc.). As described in greater detail herein, the memory 106 includes a health screening system 108 that controls performance of health assessments of users in an entertainment venue 110. Thus, the health screening system 108 is executable by the processor(s) 104. While many of the examples set forth herein show the entertainment venue 110 as including the computing system 102, it is to be appreciated that the computing system 102 (or a portion thereof) can be separate from the entertainment venue 110.

The entertainment venue 110 includes a plurality of attractions. More particularly, the entertainment venue 110 includes an attraction 1 112, . . . , and an attraction X 114, where X can be substantially any integer greater than one (collectively referred to herein as the attractions 112-114). The attractions 112-114 each include respective sensors. More particularly, the attraction 1 112 includes sensors 116, . . . , and the attraction X 114 includes sensors 118. The sensors 116 of the attraction 1 112, . . . , and the sensors 118 of the attraction X 114 are collectively referred to herein as the sensors 116-118. The sensors 116-118 of the attractions 112-114 can output signals corresponding to sensed conditions at least while users interact with the attractions 112-114.

The entertainment venue 110 can be an amusement park (e.g., theme park, etc.), for example. Thus, the attractions 112-114 can include rides (e.g., thrill rides, roller coasters, train rides, water rides, Ferris wheels, etc.), games, events, simulators (e.g., ride simulators, flight simulators, etc.), or the like. Additionally or alternatively, the attractions 112-114 can include swimming pools, other bodies of water, and/or recreational equipment. Examples of recreational equipment include equipment upon which users can run, jump, climb, swing, swim, crawl, etc. (e.g., the recreational equipment can include swings, merry-go-rounds, chin up bars, ladders, slides, etc.). For instance, a treadmill, a moving sidewalk, a stair climber, a trampoline, or the like can be part of one or more of the attractions 112-114. According to another example, the entertainment venue 110 can be a mobile facility (e.g., mobile trailer including portable recreational equipment, etc.); following this example, the attractions 112-114 can include rides, games, events, recreational equipment, etc.

The sensors 116-118 can be utilized to monitor users while such users interact with the attractions 112-114. More particularly, the sensors 116-118 of the attractions 112-114 can output signals while the users interact with the attractions 112-114 of the entertainment venue 110. According to an example, at least a portion of the sensors 116-118 can be embedded in the attractions 112-114. By way of illustration, a sensor can be embedded in a seat of a ride (e.g., the ride can be one of the attractions 112-114 of the entertainment venue 110). In accordance with another example, at least a portion of the sensors 116-118 can be physically coupled to the attractions 112-114. By way of another example, at least a portion of the sensors 116-118 can monitor parameters of the attractions 112-114. For instance, a water level of a pool of water can be monitored by a sensor (e.g., the pool can be one of the attractions 112-114 of the entertainment venue 110); a change in the water level in response to a user entering or exiting the pool can be detected based on a signal output by such sensor. Pursuant to another example, at least a portion of the sensors 116-118 can monitor environments respectively surrounding the attractions 112-114. For instance, an optical sensor (e.g., a camera) can monitor a geographic area within the entertainment venue 110 in which a game is played (e.g., the game can be one of the attractions 112-114 of the entertainment venue 110). In accordance with another example, at least a portion of the sensors 116-118 can be included or coupled to devices utilized as part of the attractions 112-114. For instance, a device may be carried as part of a game, and such device can include one or more sensors and/or have one or more sensors coupled thereto (e.g., the game can be one of the attractions 112-114 of the entertainment venue 110). It is to be appreciated, however, that the claimed subject matter is not limited to the foregoing examples.

The entertainment venue 110 can further optionally include costumes that can be worn by users. For instance, a costume 120 can be wearable by a user 122. The costume 120 can be worn by the user 122 when interacting with a particular one of the attractions 112-114 or generally while the user 122 is in the entertainment venue 110. The costume 120 can be a thematically appropriate uniform for a theme of the entertainment venue 110 or a particular one of the attractions 112-114. Examples of such types of costumes include a superhero costume, a fighter pilot flight suit, a helmet, or the like. While the system 100 shows one costume (e.g., the costume 120) in the entertainment venue 110, it is to be appreciated that the entertainment venue 110 can include substantially any number of additional costumes.

Moreover, the costume 120 includes sensors 124. The sensors 124 can be embedded in the costume 120 and/or coupled to the costume 120. One or more of the sensors 124 can output signal(s) while the user 122 wears the costume 120. Thus, the sensors 124 can output signals during interaction between the user 122 and an attraction (e.g., one of the attractions 112-114). Examples of the sensors 124 include electrical sensors (e.g., electrocardiogram (ECG) electrodes, etc.), pulse oximeters, blood pressure measurement devices, accelerometers, position sensors, breathing sensors (e.g., pressure transducers, thermocouples), microphones, pressure sensors, optical sensors, and so forth.

The health screening system 108 controls performance of a health assessment of the user 122 (as well as disparate users) in the entertainment venue 110. While many of the examples set forth herein pertain to controlling performance of a health assessment of the user 122, it is contemplated that the health screening system 108 can similarly control performance of health assessments of disparate users in the entertainment venue 110.

The health screening system 108 includes a control component 126 that controls an interaction between the user 122 and an attraction (e.g., one of the attractions 112-114) of the entertainment venue 110. The control component 126 can control the interaction between the user 122 and the attraction based on a health parameter to be measured. The interaction between the user 122 and the attraction is controlled by the control component 126 to cause one or more of the sensors in the entertainment venue 110 (e.g., one or more of the sensors 116-118 and/or the sensors 124) to more efficiently and reliably output signals that can be used to compute data indicative of the health parameter of the user 122 being measured. Moreover, a number of interactions and/or durations of such interactions between the user 122 and the attractions 112-114 of the entertainment venue 110 can be reduced by the control component 126 controlling the interaction between the user 122 and the attraction, which can reduce power consumption within the entertainment venue 110 (e.g., by eliminating unnecessary interactions and/or shortening durations of interactions). Further, network bandwidth usage due to transmission of signals output by the sensors 116-118 and/or the sensors 124 can be reduced by the control component 126 controlling the interaction between the user 122 and the attraction.

According to an example, the control component 126 can transmit a control signal to the attraction, where the control signal is configured to cause a physical modification of at least a portion of the attraction. Accordingly, the health parameter of the user can be measurable based on a response of the user 122 to the physical modification. Examples of the physical modification of the portion of the attraction that can be caused responsive to the control signal include initiating or terminating operation of the portion of the attraction (e.g., starting or stopping at least a portion of a ride), modifying operating parameters of the portion of the attraction (e.g., modifying speed, acceleration, height, depth, range of motion, orientation, angle, force, resistance, timing, duration, etc. of the portion of the attraction), altering an environmental condition for the portion of the attraction (e.g., altering temperature in a volume in which the user 122 is located, etc.), and so forth.

Additionally or alternatively, pursuant to an example, the control component 126 can select at least a portion of a storyline based on the health parameter to be measured. Further, the control component 126 can cause the portion of the storyline to be presented to the user 122 in the entertainment venue 110. Such portion of the storyline can be configured to cause the user 122 to interact with the attraction to enable the health parameter to be measured. The portion of the storyline can be output via output device(s) (e.g., display screen(s), speaker(s), projector(s), etc.), at least a portion of the attraction (e.g., movement of a robot that is part of the attraction can convey the portion of the storyline, etc.), and so forth.

The health screening system 108 further includes a collection component 128. The collection component 128 can acquire a signal output by a sensor (e.g., one of the sensors 116-118, one of the sensors 124 of the costume 120 worn by the user 122) of the attraction during the interaction between the user 122 and the attraction of the entertainment venue 110. Moreover, the collection component 128 can compute data indicative of the health parameter of the user 122 based on the signal output by the sensor. While many of the examples set forth herein describe the collection component 128 acquiring a signal output by a sensor and computing data indicative of a health parameter based on the signal, it is contemplated that these examples can be extended to scenarios where the collection component 128 acquires signals output by a plurality of sensors and computes the data indicative of the health parameter based on the signals acquired from the plurality of sensors.

The collection component 128 collects the data indicative of health parameters of the user 122 (as well as the disparate users). The data indicative of the health parameters of the user 122 can be acquired based on signals output by the sensors 116-118 and/or the sensors 124. In accordance with an illustration, the collection component 128 can receive a signal output by one of the sensors 124 of the costume 120 worn by the user 122 during the interaction between the user and the attraction. Accordingly, the collection component 128 can compute data indicative of a health parameter of the user 122 based on the signal received from the sensor 124 of the costume 120. The collection component 128 can also collect at least a portion of the data indicative of health parameters of the users from other sources. For example, the collection component 128 can receive data indicative of health parameters of the user 122 from a computing device (not shown), where the computing device receives an input that specifies such data. According to an illustration, medical personnel may evaluate the user 122 while interacting with one of the attractions 112-114 in the entertainment venue 110; based upon the evaluation, the medical personnel may input the data indicative of the health parameter to the computing device, which can send the data to the health screening system 108 (e.g., the collection component 128).

According to an illustration, the control component 126 can transmit a first control signal to a first attraction (e.g., the attraction 1 112) in the entertainment venue 110 and a second control signal to a second attraction (e.g., the attraction X 114) in the entertainment venue 110. The first control signal can be configured to cause a first physical modification of at least a portion of the first attraction. Moreover, a first health parameter of the user 122 can be measurable based on a response of the user 122 to the first physical modification. Further, the second control signal can be configured to cause a second physical modification of at least a portion of the second attraction. A second health parameter of the user 122 can be measurable based on a response of the user 122 to the second physical modification. Following this illustration, the collection component 128 can compute data indicative of the first health parameter of the user 122 based on a first signal output by a first sensor (e.g., one of the sensors 116, one of the sensors 124). The first signal can be output by the first sensor during an interaction between the user 122 and the first attraction. Moreover, the collection component 128 can compute data indicative of the second health parameter of the user 122 based on a second signal output by a second sensor (e.g., one of the sensors 118, one of the sensors 124). The second signal can be output by the second sensor during an interaction between the user 122 and the second attraction of the entertainment venue 110.

The attractions 112-114 of the entertainment venue 110 unobtrusively incorporate various medical tests to collect health data of users (e.g., the user 122, disparate users) while the users interact with the attractions 112-114. Accordingly, the users can have desirable experiences while medical testing can be performed in the entertainment venue 110. Further, users can regularly visit the entertainment venue 110 to collect personally and clinically useful health data over time.

The health screening system 108 can perform health screening via a series of entertainment experiences. Unlike conventional health screening practices, the health screening performed in the entertainment venue 110 is not conducted in a doctor's office or clinical setting, rather the user 122 visits the entertainment venue 110 and participates in a series of the attractions 112-114. The attractions 112-114 are designed with the sensors 116-118, which allow a variety of health data to be unobtrusively collected.

The entertainment venue 110 may be targeted at a specific group of users. For example, children can be particularly upset by visits to a doctor, but may be eager to visit an amusement park. Thus, a child can be brought to the entertainment venue 110, interact with a number of the attractions 112-114, and remain relatively unaware that she has completed a number of medical tests. According to another example, for adults, the desired purpose of obtaining medical health data may not be hidden, yet the goal of having an entertaining experience rather than a clinical experience can remain when visiting the entertainment venue 110.

It is contemplated that substantially any type of attraction 112-114 is intended to fall within the scope of the hereto appended claims. The following examples describe types of attractions 112-114 that may be utilized in the system 100 for health screening in the context of entertainment attractions. Yet, it is to be appreciated that the claimed subject matter is not so limited.

By way of example, one of the attractions 112-114 can be a roller coaster with sensors embedded in and/or coupled to a seat or a restraint. The user 122 can sit in the seat and be restrained by the restraint while the roller coaster is operational. Accordingly, the sensors of the roller coaster can output signals indicative of various health parameters of the user 122 while the user 122 undergoes the stress of the roller coaster.

According to another example, one of the attractions 112-114 may be a motion ride that inverts the user 122. Accordingly, sensors of such ride can enable measurement of blood flow while the position, orientation, speed, and/or acceleration of the user 122 is manipulated (e.g., inversion of the user 122, differing amounts of rotation of the user 122, traveling at different speeds, accelerating, decelerating, etc.).

By way of yet another example, one of the attractions 112-114 can provide an entertaining context for patient-required movement. By way of illustration, a cardiac stress test may involve the user 122 running from a villain. The attraction, for instance, may include a moving walkway, a treadmill, or the like upon which the user 122 runs within the entertainment venue 110. Alternatively, the user 122 may run through the entertainment venue 110. Accordingly, the sensors of such attraction can output signals indicative of health parameters while the user 122 is running from such villain.

Pursuant to another example, one of the attractions 112-114 can be a simulation game that can incorporate game mechanics that test hearing, vision, touch sensation, reaction times, or the like, of the user 122. In accordance with yet another example, one of the attractions 112-114 can be a pool of water. Following this example, the user 122 can enter the pool of water. Immersion in the water as part of an activity can allow for body volume, lung capacity, body fat, and so forth of the user 122 to be measured.

In accordance with yet another example, one of the attractions 112-114 can include an invasive test. A story can be built around such invasive test. Accordingly, costumed medical personnel can perform dual roles as actors and medical providers. Health data collected by a medical provider during the invasive test as part of a storyline can further be inputted to the health screening system 108 (e.g., via a computing device of the medical provider, etc.). Moreover, medical testing equipment used by the medical personnel can additionally or alternatively provide results of a medical test to the health screening system 108.

Pursuant to various examples, one or more of the attractions 112-114 can modify an activity level of the user 122, a posture of the user 122 (e.g., the user 122 can lay down, sit, stand, dangle upside down, or change between such positions), and/or a temperature of the user 122. Further, portion(s) of at least one of the attractions 112-114 can enable assessment of reaction time, range of physical motion, strength, nerve response, a combination thereof, and so forth.

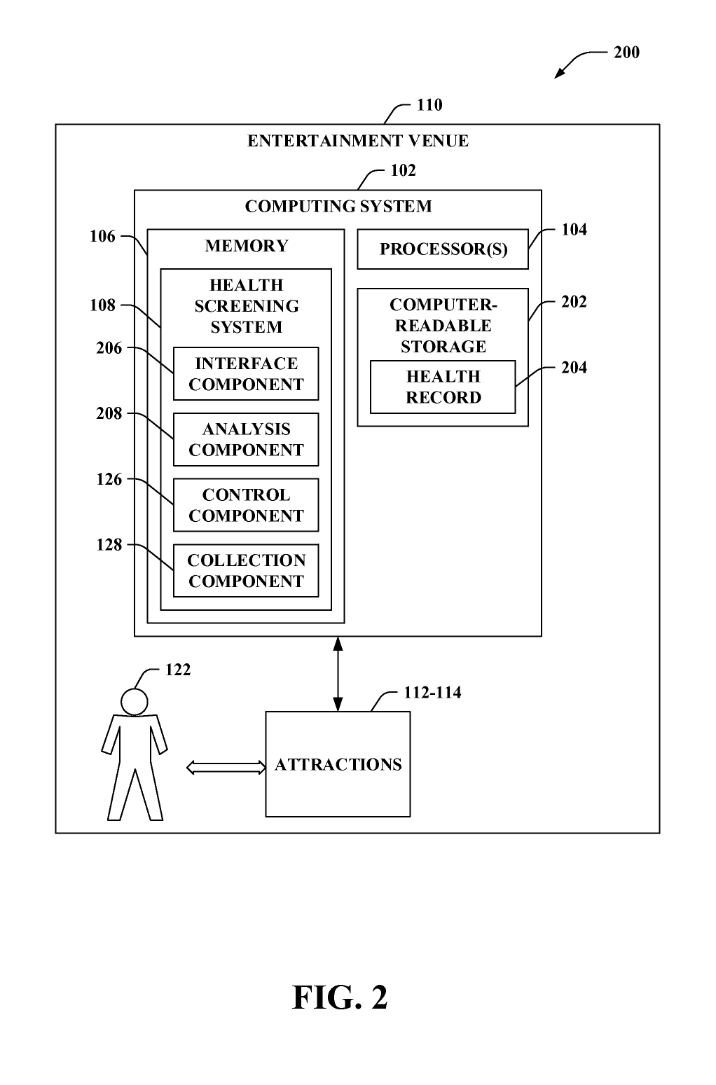

Turning to FIG. 2, illustrated is a system 200 that controls performance of a health assessment of the user 122 in the entertainment venue 110. The system 200 again includes the computing system 102. As depicted in FIG. 2, the computing system 102 includes computer-readable storage 202. While not shown, according to various examples, it is contemplated that the computer-readable storage 202 can be or include the memory 106.

The computer-readable storage 202 can retain a health record 204 of the user 122. The computer-readable storage 202 can also retain health records of disparate users. The health record 204 can include data indicative of health parameters of the user 122. For instance, at least a portion of the data indicative of the health parameters of the user 122 included in the health record 204 can be acquired by the collection component 128. The health screening system 108 can aggregate data indicative of health parameters of the user 122 in the health record 204 of the user 122. Moreover, the health screening system 108 can similarly aggregate data indicative of health parameters of disparate users in corresponding health records of the disparate users.

The health screening system 108 can include an interface component 206 that accesses data in the health record 204 of the user 122. The interface component 206 can similarly access data in health records of disparate users. The data in the health record 204 can be written, read, or modified by the health screening system 108 via the interface component 206.

The health screening system 108 further includes an analysis component 208 that selects a health parameter of the user to be measured as part of the health assessment performed in the entertainment venue 110. The health parameter can be selected by the analysis component 208 based on the data in the health record 204 of the user 122. Accordingly, the control component 126 can control an interaction between the user 122 and an attraction (e.g., one of the attractions 112-114) of the entertainment venue 110 based on the health parameter to be measured. The control component 126 can further control the interaction between the user 122 and the attraction based on the data in the health record 204 of the user 122. Further, the collection component 128 can compute data indicative of the health parameter of the user 122 based on a signal output by a sensor (e.g., one of the sensors 116-118, one of the sensors 124), where the signal is output by the sensor during the interaction between the user 122 and the attraction of the entertainment venue 110. Moreover, the interface component 206 can store the data indicative of the health parameter of the user 122 in the health record 204 of the user 122.

Data indicative of a second health parameter of the user 122 measured during an interaction between the user 122 and a second attraction (e.g., a differing one of the attractions 112-114) of the entertainment venue 110 can similarly be acquired and stored in the health record 204 of the user 122. Similar to above, the second health parameter of the user 122 can be selected to be measured as part of the health assessment by the analysis component 208. Further, the interaction between the user 122 and the second attraction can be controlled by the control component 126 based on the second health parameter to be measured. The collection component 128 can compute the data indicative of the second health parameter of the user 122 based on a signal output by a sensor (e.g., one of the sensors 116-118, one of the sensors 124), where the signal is output by the sensor during the second interaction between the user 122 and the second attraction of the entertainment venue 110. Moreover, the interface component 206 can store the data indicative of the second health parameter of the user 122 in the health record 204 of the user 122. It is to be appreciated that substantially any number of additional health parameters of the user 122 can similarly be acquired and stored in the health record 204 of the user 122.

The analysis component 208 evaluates the health record 204 of the user 122. The analysis component 208, for example, can detect conditions from the health record 204 of the user 122. According to other examples, the analysis component 208 can detect inconsistencies in the health record 204, data missing from the health record 204 (e.g., the user 122 has yet to have a certain type of test performed, etc.), or the like. Further, the analysis component 208 can similarly evaluate health records of disparate users.

The analysis component 208 can select a health parameter (or a sequence of health parameters) of the user 122 to be measured as part of a health assessment performed in the entertainment venue 110. Based on the selected health parameter(s), interaction(s) with one or more of the attractions 112-114 in the entertainment venue 110 can be planned and/or suggested (e.g., to the user 122). For instance, the control component 126 can control the interaction(s) with the attraction(s) 112-114 based on the health parameters selected by the analysis component 208. Planning or suggesting the attractions 112-114 with which the user 122 is to interact can lead to reduced energy consumption by the attractions 112-114 of the entertainment venue 110 (e.g., unneeded interactions for the user 122 are not planned or suggested, which leads to reduction in interactions with the attractions 112-114). Further, planning or suggesting the attractions 112-114 with which the user 122 is to interact can lead to reduced network bandwidth usage due to reduced transmission of signals from sensors for unneeded interactions for the user 122.

The analysis component 208 can generate a suggestion of an attraction from the attractions 112-114 with which the user 122 is recommended to interact. For instance, the analysis component 208 can evaluate the health record 204 of the user 122 to determine health data of the user 122 that is missing (e.g., blood pressure of the user 122 has yet to be measured by the health screening system 108, etc.). Based on the missing health data in the health record 204, the analysis component 208 can generate a suggestion for the user 122 to interact with an attraction from the attractions 112-114 that can capture such health data from the user 122 during the user's interaction with such attraction in the entertainment venue 110. According to another example, the analysis component 208 can generate a suggestion for an attraction based on a condition being monitored by the health screening system 108 for the user 122 (e.g., a heart condition of the user 122 is being monitored, etc.). Pursuant to yet another example, the analysis component 208 can suggest an attraction due to variances between health data of the user 122 collected by attractions (e.g., two or more attractions collect inconsistent health data of the user 122); thus, the analysis component 208 can generate a suggestion that the user 122 interact with an attraction that can capture such health data of the user 122 to resolve the inconsistency. Moreover, it is to be appreciated that the analysis component 208 can similarly generate a suggestion that a costume be worn by the user 122 in the entertainment venue 110 (e.g., the costume can be worn generally in the entertainment venue 110 or worn for a specific attraction, etc.).

Further, the analysis component 208 can create an order of the attractions 112-114 for the user 122 to capture user health data in an appropriate sequence. Thus, the analysis component 208 can create a sequence that sets a first attraction from the attractions 112-114 suggested for the user 122, followed by a second attraction from the attractions 112-114, and so forth. The sequence of the attractions 112-114 can be formed by the analysis component 208 based on a condition being monitored, for example; yet, the claimed subject matter is not so limited.

A suggestion generated by the analysis component 208 can be used by the control component 126. For example, the control component 126 can cause the user 122 to be directed towards a suggested interaction with a particular attraction. By way of illustration, the control component 126 can cause a storyline presented to the user 122 to be tailored to direct the user 122 to interact with the particular attraction.

Moreover, returning users can access the history of their health records (e.g., the user 122 can return to the entertainment venue 110 at a later date and again access the health record 1204) to allow for vital statistics to be tracked over time. According to another example, the health record 204 can be deleted from the computer-readable storage 202 subsequent to the health record 204 being sent to the user 122 (e.g., a computing device of the user 122, a device of the user 122 that includes computer-readable memory, etc.). For instance, the health record 204 can be deleted from the computer-readable storage 202 upon the user 122 leaving the entertainment venue 110. Following this example, the user 122 can later return to the entertainment venue 110 and supply historical health record(s), which can be imported into the health screening system 108 to allow for vital statistics to be tracked over time. Deletion of the health record 204 upon the user 122 leaving the entertainment venue 110 leads to enhanced data security for the health screening system 108, since the data of the health record 204 is stored in the computer-readable storage 202 only while the user 122 is at the entertainment venue 110.

According to an example, one or more of the attractions 112-114 can be group attractions. As part of a group attraction, the user 122 and a disparate user (or plurality of disparate users) concurrently interact with the attraction. For instance, the analysis component 208 can select a health parameter of the user 122 to be measured and a health parameter of the disparate user to be measured. The control component 126 can concurrently control an interaction between the user 122 and the attraction based on the health parameter of the user 122 to be measured and an interaction between the disparate user and the attraction based on the health parameter of the disparate user to be measured. Further, the collection component 128 can compute data indicative of the health parameter of the user based on a signal output by a sensor, where the signal is output by the sensor during the interaction between the user and the attraction. Moreover, the collection component 128 can compute data indicative of the health parameter of the disparate user based on a signal output by a disparate sensor, where the signal is output by the disparate sensor during the interaction between the disparate user and the attraction.

A roller coaster is an example of a group attraction. The control component 126 can cause a first user to sit in a first seat of the roller coaster and a second user to sit in a second seat of the roller coaster (e.g., a storyline may direct the users to their respective seats, etc.). The differing seats of the roller coaster can provide differing experiences for the users (e.g., the users can be subjected to different movements, forces, etc.), which can enable different health parameters to be measured.

Another example of a group attraction is an attraction where a monster chases two or more users. The users may each run on their own treadmill; the speed and/or angle of incline of each of the treadmills can be independently controlled by the control component 126. Thus, the users are collectively experiencing the attraction as a group, but operating parameters of the treadmills are separately controlled to enable health parameters of the users to be individually assessed.

Yet another example of a group attraction is a raft ride simulator. Two or more users can simultaneous interact with the simulator; however, amounts of movement for each of the users can be separately controlled by the control component 126.

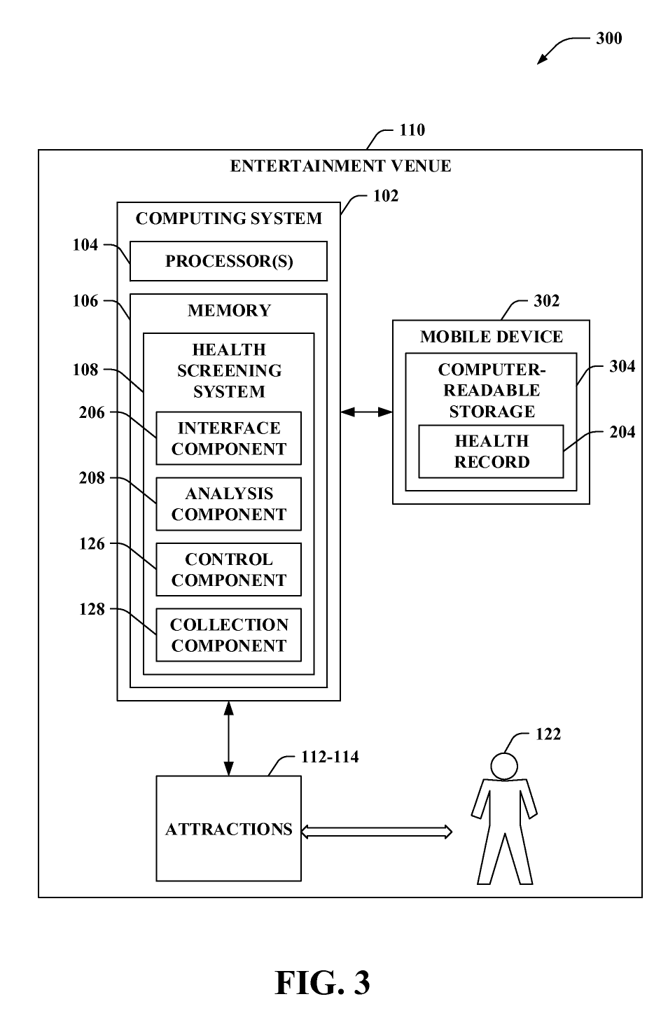

Turning to FIG. 3, illustrated is another system 300 that controls performance of a health assessment of the user 122 in the entertainment venue 110. The system 300 includes the computing system 102 and a mobile device 302. In the example set forth in FIG. 3, the mobile device 302 includes computer-readable storage 304 that retains the health record 204 of the user 122. Thus, the mobile device 302 with the health record 204 retained in the computer-readable storage 304 can be brought into the entertainment venue 110 (e.g., by the user 122, along with the user 122). Load on the processor(s) 104 is reduced by using data in the health record 204 retained in the computer-readable storage 304 of the mobile device 302 as compared to recreating such data responsive to the user 122 returning to the entertainment venue 110.

Examples of the mobile device 302 include a laptop computing device, a mobile telephone (e.g., smartphone), a tablet computing device, a wearable computing device, a handheld computing device, a portable gaming device, a personal digital assistant, or the like. According to other examples, the mobile device 302 can be a portable storage device.

Again, the interface component 206 of the health screening system 108 can access data in the health record 204 retained in the computer-readable storage 304 of the mobile device 302. Moreover, the interface component 206 can store data indicative of health parameters of user 122 (e.g., acquired by the health screening system 108) in the health record 204 retained in the computer-readable storage 304 of the mobile device 302. Thus, pursuant to the example set forth in FIG. 3, the computing system 102 need not retain the health record 204 of the user 122. Further, security of the health record 204 can be improved by the health record 204 being stored in the computer-readable storage 304 of the mobile device 302.

According to another example, it is contemplated that the health record 204 (or a portion thereof) can be retrieved by the interface component 206 from the computer-readable storage 304 of the mobile device 302 and retained in computer-readable storage (e.g., the computer-readable storage 202) of the computing system 102 for utilization while the user 122 is located at the entertainment venue 110. For instance, upon the user 122 entering the entertainment venue 110, the interface component 206 can retrieve the health record 204 (or the portion thereof) of the user 122 from the computer-readable storage 304 of the mobile device 302. While the user 122 interacts with the attractions 112-114 during a visit at the entertainment venue 110, data can be read, written, or modified in a copy of the health record 204 maintained in the computer-readable storage 202 of the computing system 102. Thereafter, upon the user 122 leaving the entertainment venue 110, the health record 204 retained in the computer-readable storage 304 of the mobile device 302 can be updated based on changes to the copy of the health record 204 maintained in the computer-readable storage 202 of the computing system 102. Moreover, it is contemplated that the copy of the health record 204 maintained in the computer-readable storage 202 of the computing system 102 can be deleted in response to the user 122 leaving the entertainment venue 110. Pursuant to yet another example, the health record 204 (or a portion thereof) can similarly be retrieved from the computer-readable storage 304 of the mobile device 302 and retained in the computer-readable storage 202 of the computing system 102 for utilization while the user 122 interacts with an attraction; following this example, upon the interaction with the attraction ending, the health record 204 retained in the computer-readable storage 304 of the mobile device 302 can be updated and the copy of the health record 204 maintained in the computer-readable storage 202 of the computing system 102 can be deleted.

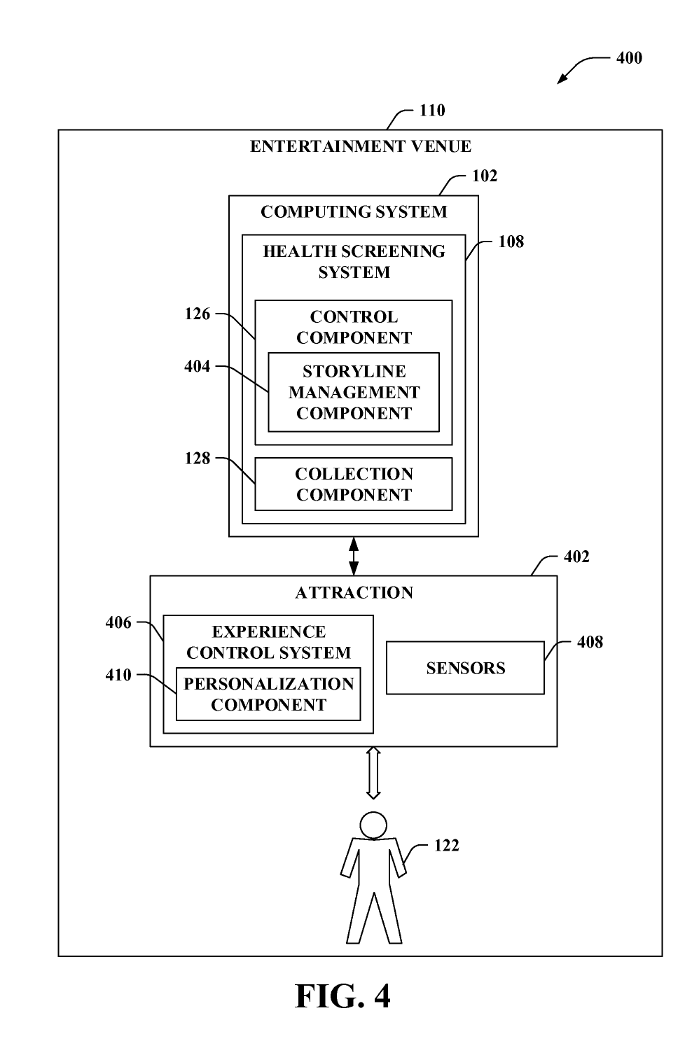

With reference to FIG. 4, illustrated is a system 400 that tailors an experience of the user 122 when interacting with an attraction 402 (e.g., one of the attractions 112-114) in the entertainment venue 110. The control component 126 of the health screening system 108 controls an interaction between the user 122 and the attraction 402 based on a health parameter to be measured. In the example set forth in FIG. 4, the control component 126 can further include a storyline management component 404. The storyline management component 404 selects at least a portion of a storyline based on the health parameter to be measured. The storyline management component 404 can further cause the portion of the storyline to be presented to the user 122 in the entertainment venue 110. The portion of the storyline can be configured to cause the user 122 to interact with the attraction 402 to enable the health parameter to be measured. For instance, the portion of the storyline can be presented via display screen(s), speaker(s), projector(s), or substantially any other type of output device(s) in the entertainment venue 110. Additionally or alternatively, at least a portion of the attraction 402 can convey the portion of the storyline (e.g., through movement of the portion of the attraction 402, etc.).

The attraction 402 can include an experience control system 406 that manages the attraction 402. For instance, the experience control system 406 can manage a speed of the attraction 402, an angle of the attraction 402, a length of time that the user 122 interacts with the attraction 402, images displayed on a display screen(s) included in the attraction 402, sound output by speaker(s) as part of the attraction 402, or substantially any other operating parameter of the attraction 402. The experience control system 406 can manage the attraction 402 responsive to a control signal received from the control component 126 of the health screening system 108, for example. The experience control system 406 can physically modify at least a portion of the attraction 402 responsive to the control signal. The attraction 402 further includes sensors 408 that output signals during the interaction between the attraction 402 and the user 122; the collection component 128 can receive the signals from the sensors 408 and compute data indicative of health parameter(s) of the user 122 based upon such signals.

The storyline management component 404 can modify the storyline utilized by the attraction 402. The storyline can be implemented by the experience control system 406. The storyline management component 404, for instance, can modify the storyline for the user 122 based on characteristics of the user (e.g., age, weight, height, chronic medical conditions, etc.). Moreover, the storyline management component 404 can modify the storyline based on conditions detected from the health record 204 of the user 122, missing or inconsistent health data in the health record of the user 122, or the like.

The experience control system 406 can include a personalization component 410 that can receive the tailored storyline from the health screening system 108 (e.g., from the storyline management component 404). The personalization component 410 can further control the experience control system 406 to manage operation of the attraction 402 based on the storyline as tailored to the user 122 by the storyline management component 404.

The interaction between the user and the attraction 402 of the entertainment venue 110 can be controlled by the control component 126 to perform a cardiac stress test on the user 122. The control component 126 can transmit a control signal to the attraction 402. The control signal can be configured to cause a physical modification of at least a portion of the attraction 402. Thus, the experience control system 406 can physically modify the portion of the attraction 402 responsive to the control signal. The physical modification of the portion of the attraction can be configured to induce stress on the user 122. The physical modification of the portion of the attraction 402 can induce the user 122 to exercise (e.g., cause the user 122 to jog, run, jump, swim, lift, push, pull, etc.). Moreover, data of the cardiac stress test can be measured by the collection component 128 before, during, and after induction of the stress on the user 122 based on signal(s) output by one or more of the sensors 408 (and/or sensors of a costume worn by the user 122 when interacting with the attraction 402). For the cardiac stress test, signal(s) output by sensors such as an electrocardiogram (ECG) device, a blood pressure measurement device, an ultrasound scanner, an x-ray device, a magnetic resonance imaging (MRI) scanner, a plurality thereof, and so forth can be used to measure the data of the cardiac stress test. Thus, in the entertainment venue 110, a level of stress can be induced on the user 122 while the user 122 is entertained, and the data of the cardiac stress test can be measured with sensors that are part of the attraction 402 and/or the costume worn by the user 122 as part of the experience.

Further, the interaction between the user and the attraction 402 of the entertainment venue 110 can be controlled to perform a sleep test on the user 122. The control component 126 can cause a portion of a storyline to be presented to the user 122 in the entertainment venue 122; thus, the experience control system 406 can present the portion of the storyline to the user 122. The portion of the storyline can be configured to prompt the user to sleep in the attraction 402 (e.g., the attraction 402 can include a bed, a room designated for the user 122, etc.). Moreover, the collection component 128 can measure data of the sleep test while the user 122 sleeps in the attraction 402 based on the signal(s) output by one or more of the sensors 408 (and/or the sensors of the costume worn by the user 122 when interacting with the attraction 402). For the sleep test, signal(s) output by sensors such as an electroencephalogram (EEG) device, an electrooculogram (EOG) device, an electromyogram (EMG) device, an electrocardiogram (ECG) device, a pressure transducer, a thermocouple, a pulse oximeter, a microphone, a plurality thereof, and so forth can be used to measure the data of the sleep test. While the user 122 sleeps in the attraction 402, the EEG device can be used to measure brain activity, the EOG device can be used to measure eye movement, the EMG device can be used to monitor muscle activity or skeletal muscle activation, the ECG device can be used to monitor heart rhythm, the pressure transducer and/or the thermocouple can be used to measure nasal and oral airflow, the pulse oximeter can be used to monitor blood oxygen levels, and/or the microphone can be used to record snoring.

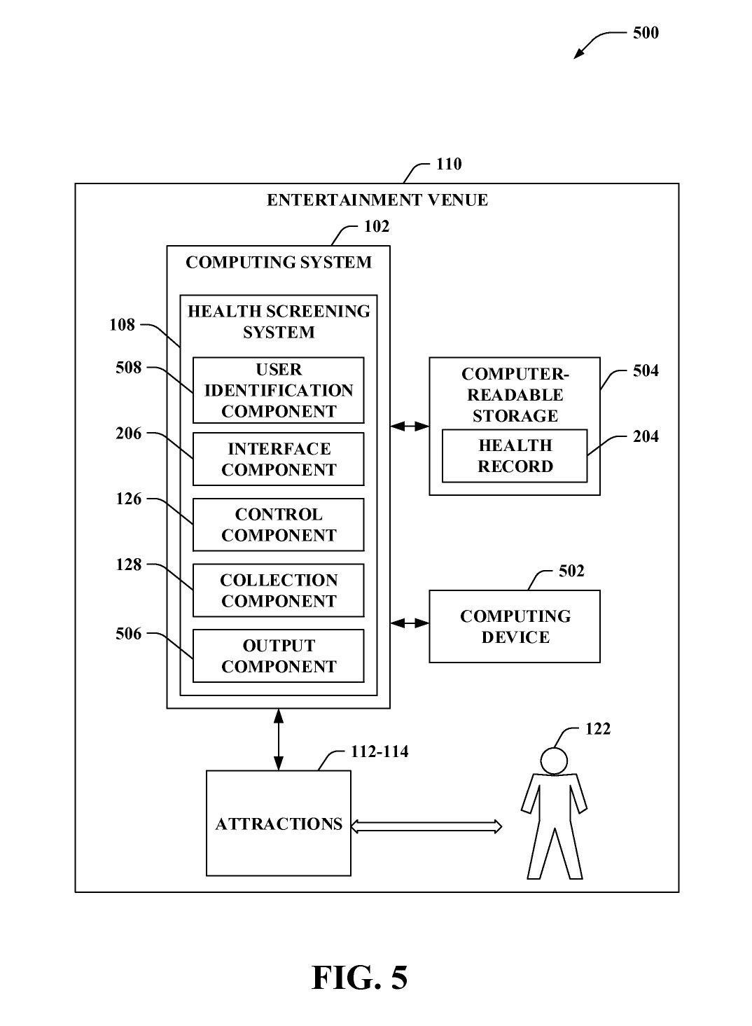

With reference to FIG. 5, illustrated is a system 500 that distributes data from the health record 204 of the user 122 to a computing device 502. The health record 204 can be retained in computer-readable storage 504 (e.g., the computer-readable storage 202 of the computing system 102 and/or the computer-readable storage 304 of the mobile device 302). Moreover, while the computing device 502 is shown as being included in the entertainment venue 110 (e.g., located at the entertainment venue 110), it is to be appreciated that the computing device 502 can be outside of the entertainment venue 110.

As depicted in FIG. 5, the health screening system 108 includes the interface component 206 that accesses the health record 204 of the user 122 retained in the computer-readable storage 504. The interface component 206 can store data indicative of a health parameter of the user 122 in the health record 204 of the user 122. The health screening system 108 can further include an output component 506 that outputs the health record 204 of the user 122 from the health screening system 108. According to an example, the output component 506 can receive a request for the health record 204 of the user 122 (e.g., the request can be received from the computing device 502 or a disparate computing device). Responsive to the request, the output component 506 can transmit the health record 204 of the user 122 to the computing device 502.

According to an example, at an end of a session of the user 122 at the entertainment venue 110, the output component 506 can output a report that includes at least a portion of the health record 204 to the user 122 or a legal guardian of the user 122; thus, the report can be transmitted by the output component 506 to the computing device 502 (e.g., the computing device 502 can present the report to the user 122 or the legal guardian). Such report can include collected data obtained by the collection component 128 while the user 122 interacts with the attractions 112-114 (or a subset thereof) of the entertainment venue 110. Pursuant to an illustration, the computing device 502 can be the mobile device 302 of FIG. 3; yet, the claimed subject matter is not so limited.

By way of another example, the output component 506 can receive a request to send the health record 204 of the user 122 to a doctor for review. Thus, the output component 506, responsive to such request, can send the health record 204 of the user 122 to the doctor (e.g., to the computing device 502 which can be used by the doctor to review the health record 204).

Optionally, medical professionals can be onsite at the entertainment venue 110. Accordingly, the output component 506 can send the health record 204 to such medical professionals via the computing device 502 for onsite counseling to assist interpreting the health record 204 of the user 122.

Storage and distribution of the health record 204 is secured. The health record 204 may be highly sensitive (e.g., subject to various privacy regulations such as the Health Information Portability and Accountability Act (HIPAA)). Therefore, the health record 204 is stored on highly secured server(s) (e.g., the computer-readable storage 504) with restricted access (e.g., physical access to the server(s) is restricted, access to data on the server(s) is be restricted, etc.). Moreover, the health record 204 is encrypted when transmitted by the output component 506, etc.

Moreover, the health screening system 108 can optionally include a user identification component 508 that identifies the user 122 located in the entertainment venue 110. The user identification component 508 can also identify other users in the entertainment venue 110. According to various examples, the user identification component 508 can identify the user 122 based upon a token carried by the user 122 (e.g., an armband, a necklace, a card, a badge, etc.), a costume worn by the user 122, biometric characteristics sensed from the user 122, an image of the user 122, a gesture of the user 122, a personal identifier supplied by the user 122, or the like. According to yet another example, the user identification component 508 can identify the user 122 based upon a mobile device (e.g., the mobile device 302) carried by the user 122.

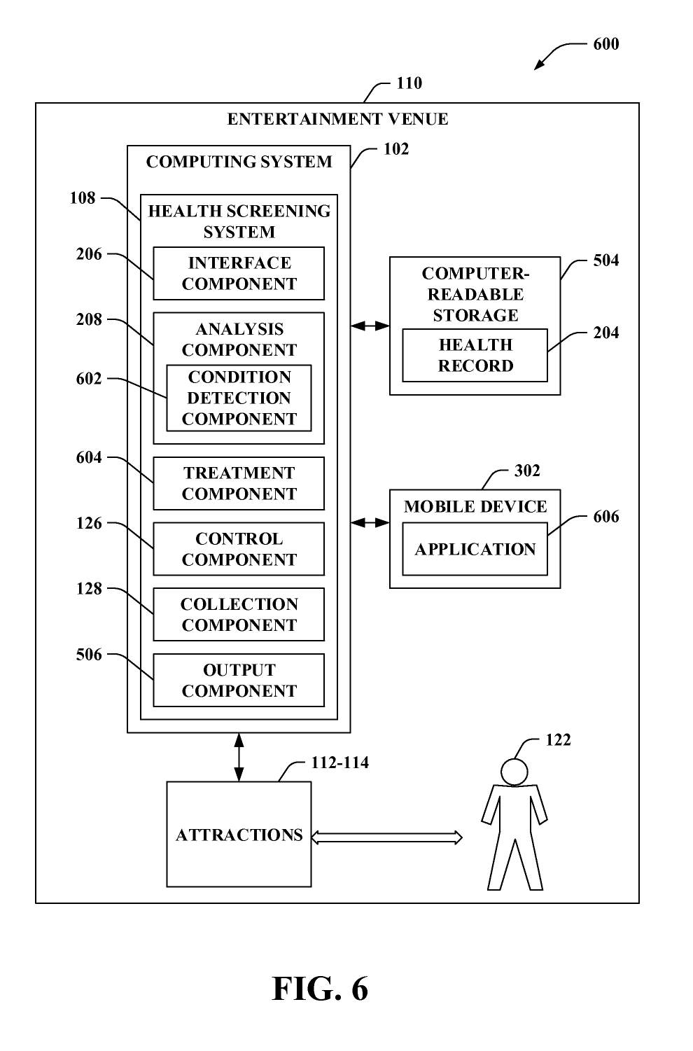

Referring now to FIG. 6, illustrated is a system 600 that controls personalization of treatment based on data collected from the user 122 at the entertainment venue 110. The system 600 includes the computing system 102. Further, the computing system 102 includes the health screening system 108.

The health screening system 108 can include the control component 126 that controls an interaction between the user 122 and an attraction (e.g., one of the attractions 112-114) of the entertainment venue 110. Moreover, the health screening system 108 includes the collection component 128 that acquires a signal output by a sensor during the interaction between the user 122 and the attraction of the entertainment venue 110. Further, the collection component 128 computes data indicative of a health parameter of the user 122 based on the signal output by the sensor. The health screening system 108 can also include the interface component 206 that stores the data indicative of the health parameter of the user 122 in the health record 204 of the user 122 retained in the computer-readable storage 504.

Moreover, the health screening system 108 includes the analysis component 208 that analyzes heath records assembled by the health screening system 108 in the entertainment venue 110. The analysis component 208 can further include a condition detection component 602. The condition detection component 602 can detect a health condition of the user 122 based at least in part upon the data indicative of the health parameter of the user 122. Further, the condition detection component 602 can detect the health condition of the user 122 based on the health record 204 of the user 122.

For example, the condition detection component 602 can compare health data values (e.g., blood pressure, body fat, heart rate, heart rate variability, etc.) from the health record 204 of the user 122 to predefined ranges. Accordingly, if a health data value included in the health record 204 is recognized to be outside of a normal (e.g., healthy) range, then the condition detection component 602 can detect a health condition of the user 122.

The health screening system 108 further includes a treatment component 604 that generates treatment plan data for the user 122 based on the health condition of the user 122. Moreover, the output component 506 can transmit the treatment plan data to the mobile device 302 of the user 122.

The mobile device 302 can execute an application 606. The application 606 can be personalized for the user 122 based on the treatment plan data received from the health screening system 108. The treatment plan data, for instance, can cause the application 606 to output a health suggestion via the mobile device 302. Examples of the health suggestion include a workout regimen, a prompt for an activity to be performed by the user 122, a grocery list, goals for the user 122, targets for the user 122, and so forth.

Pursuant to an example, the control component 126 can include the storyline management component 404. The storyline management component 404 can cause a storyline to be presented to the user 122 during the interaction between the user 122 and the attraction of the entertainment venue 110. The application 606, when executed by the mobile device 302, can continue to cause the storyline to be presented to the user 122. Thus, an experience created by the application 606 executed by the mobile device 302 can tie in with an experience in the entertainment venue 110, while providing personalized suggestions and/or being prepopulated with measurables collected in the entertainment venue 110.

By way of another example, the output component 506 can deliver health information (e.g., the treatment plan data) in the context of an attraction. For instance, the storyline management component 404 can script the storyline to output the health information to the user 122 in the context of the attraction.

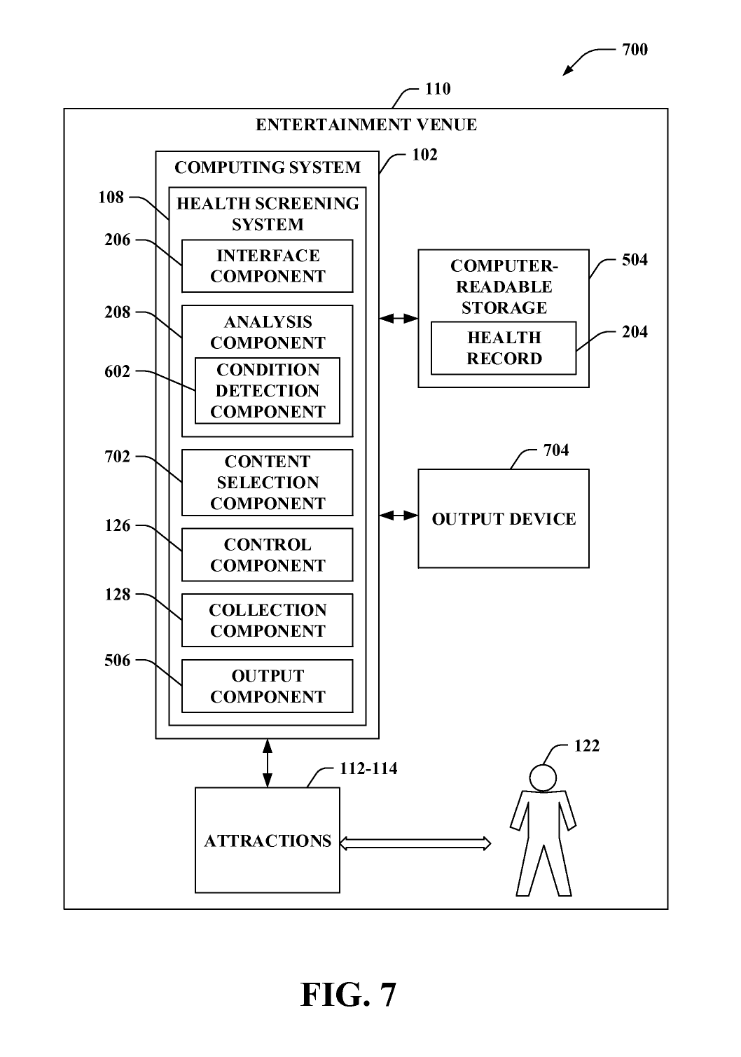

With reference to FIG. 7, illustrated is a system 700 that controls personalization of an educational experience for the user 122 based on a detected condition. Similar to FIG. 6, the health screening system 108 can include the interface component 206, the analysis component 208, the control component 126, the collection component 128, and the output component 506. Moreover, the analysis component 208 can include the condition detection component 602 that detects a health condition of the user 122 based at least in part upon the data indicative of the health parameter of the user 122. The condition detection component 602 can further detect the health condition of the user 122 based at least in part upon data included in the health record 204 of the user 122.

The health screening system 108 can further include a content selection component 702 that selects educational data for the user 122 based on the health condition of the user 122. Moreover, the output component 506 can present the educational data to the user 122. For instance, the output component 506 can send the educational data to an output device 704, which can output the educational data to the user 122. The output device 704, for example, can be the mobile device 302 of the user 122. According to another example, the output device 704 can be a display, a speaker, a projector, or the like.

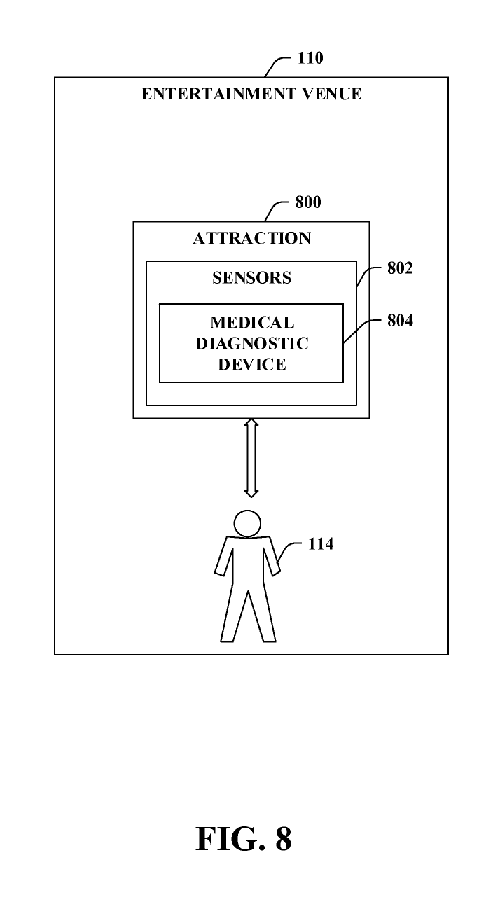

With reference to FIG. 8, illustrated is an exemplary attraction 800 of the entertainment venue 110. The attraction 800 is one of the attractions 112-114 (e.g., the attraction 800 can be the attraction 402 of FIG. 4). Although not shown, it is contemplated that the attraction 800 can include the experience control system 406 as described above.

As described herein, the attraction 800 includes sensors 802 (e.g., the sensors 116-118, the sensors 408). The sensors 802 can include a medical diagnostic device 804. Although not shown, it is contemplated that the sensors 802 of the attraction 800 can include more than one medical diagnostic device 804. The medical diagnostic device 804 can be part of a storyline implemented by the attraction 800. By way of illustration, the medical diagnostic device 804 can be a magnetic resonance imaging (MRI) scanner. Following this illustration, the MRI scanner can be themed as a time machine as part of the attraction 800, where such time machine transports a user as part of a time traveling journey in the storyline.

Other examples of the medical diagnostic device 804 include an electrocardiogram (ECG) device, a blood pressure measurement device, an ultrasound scanner, an x-ray device, a computed tomography (CT) scanner, an electroencephalogram (EEG) device, an electrooculogram (EOG) device, an electromyogram (EMG) device, a heart rate monitor, a pulse oximeter, and a breathing sensor (e.g., a pressure transducer, a thermocouple). However, substantially any other type of the medical diagnostic device 804 is intended to fall within the scope of the hereto appended claims. Further, the attraction 800 can include a plurality of different types of medical diagnostic devices. Moreover, the sensors 802 can include other types of sensors such as a camera, a pressure sensor, an electrical sensor, a microphone, and so forth. It is contemplated that the claimed subject matter is not limited to the foregoing examples, and any other types of sensors are intended to fall within the scope of the hereto appended claims.

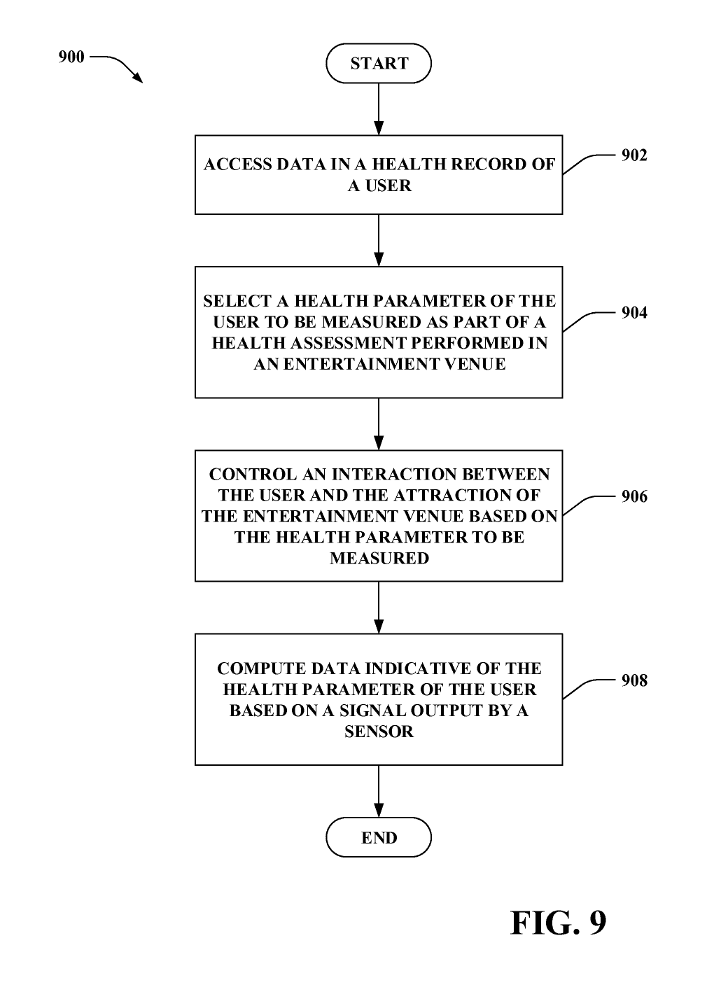

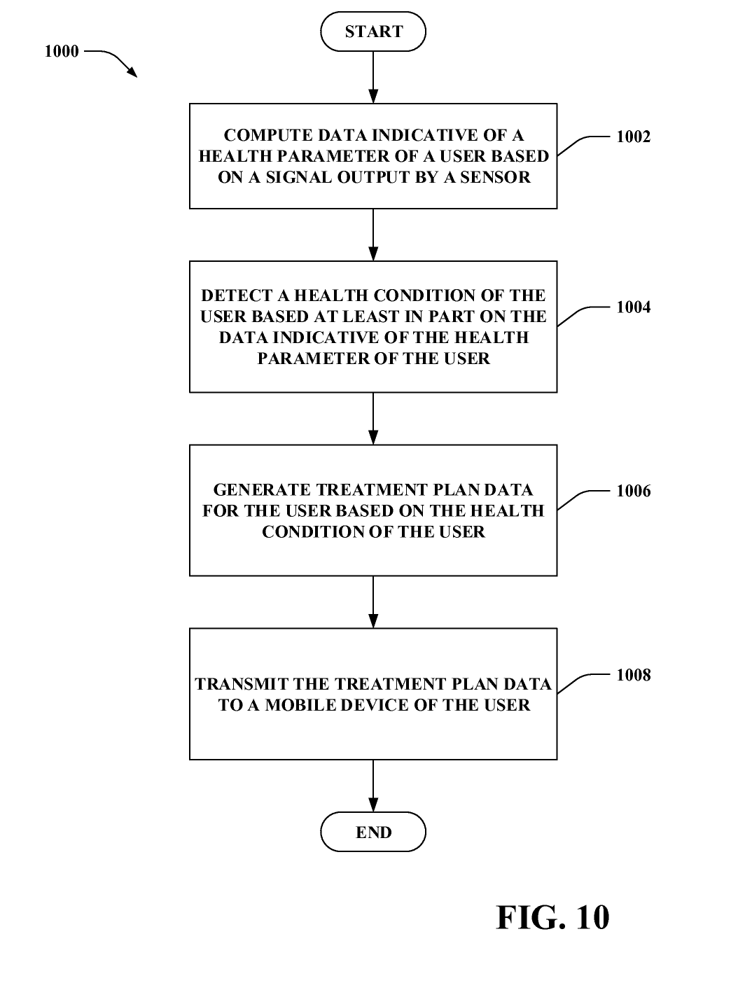

FIGS. 9-10 illustrate exemplary methodologies relating to providing health screening via an entertainment experience. While the methodologies are shown and described as being a series of acts that are performed in a sequence, it is to be understood and appreciated that the methodologies are not limited by the order of the sequence. For example, some acts can occur in a different order than what is described herein. In addition, an act can occur concurrently with another act. Further, in some instances, not all acts may be required to implement a methodology described herein.

Moreover, the acts described herein may be computer-executable instructions that can be implemented by one or more processors and/or stored on a computer-readable medium or media. The computer-executable instructions can include a routine, a sub-routine, programs, a thread of execution, and/or the like. Still further, results of acts of the methodologies can be stored in a computer-readable medium, displayed on a display device, and/or the like.

FIG. 9 illustrates a methodology 900 of controlling performance of a health assessment of a user in an entertainment venue. At 902, data in a health record of the user can be accessed. The health record can be retained in computer-readable storage. Further, the user can be located at the entertainment venue, and the entertainment venue includes an attraction. At 904, a health parameter of the user is selected to be measured as part of a health assessment performed in the entertainment venue. The health parameter can be selected based on the data in the health record of the user. At 906, an interaction between the user and the attraction of the entertainment venue can be controlled based on the health parameter to be measured. At 908, data indicative of the health parameter of the user can be computed based on a signal output by a sensor. The signal can be output by the sensor during the interaction between the user and the attraction of the entertainment venue.

With reference to FIG. 10, illustrated is a methodology 1000 of controlling personalization of treatment of a user detected to have a health condition in an entertainment venue. At 1002, data indicative of a health parameter of the user can be computed based on a signal output by a sensor. The signal can be output by the sensor during an interaction between the user and an attraction of an entertainment venue, with the user being located at the entertainment venue. At 1004, the health condition of the user can be detected based at least in part on the data indicative of the health parameter of the user. At 1006, treatment plan data for the user can be generated based on the health condition of the user. At 1008, the treatment plan data can be transmitted to a mobile device of the user. An application executable by the mobile device can be personalized for the user based on the treatment plan data.

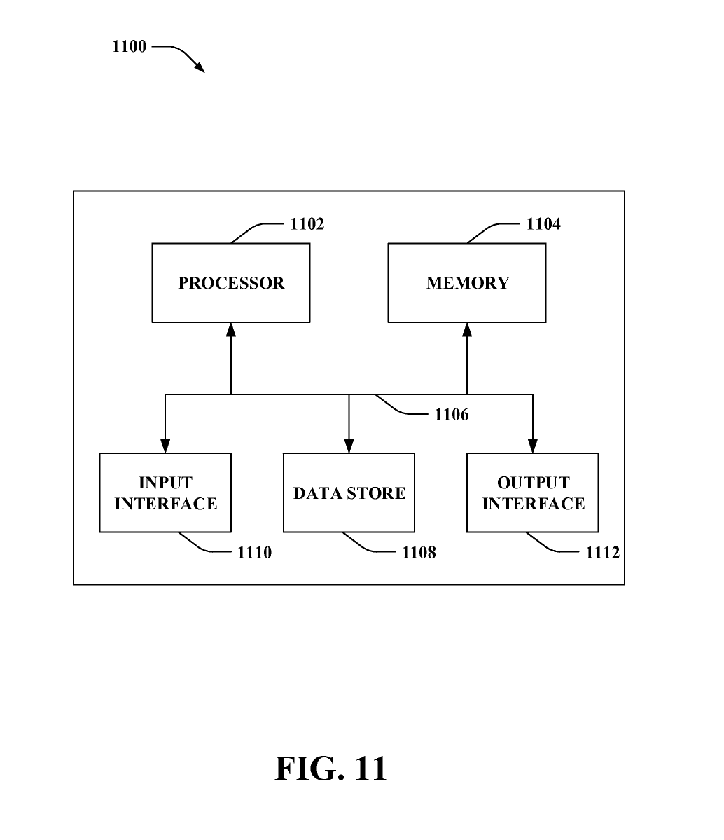

Referring now to FIG. 11, a high-level illustration of an exemplary computing device 1100 that can be used in accordance with the systems and methodologies disclosed herein is illustrated. For instance, the computing device 1100 may be or include the computing system 102. Pursuant to another example, the computing system 102 may include the computing device 1100. According to yet another example, an attraction or costume may include the computing device 1100. In accordance with yet another example, the computing device 1100 may receive input (e.g., from medical personnel that monitor a user interacting with an attraction in the entertainment venue, from a medical diagnostic device or other sensor in the entertainment venue, from testing equipment that evaluates a sample obtained from a user during interaction with an attraction of the entertainment venue, etc.); further, the received input can be sent by the computing device 1100 to the computing system 102. By way of yet a further example, the computing device 1100 may receive output from the computing system 102. According to another example, the computing device 1100 can control an attraction. The computing device 1100, for example, can be or include the mobile device 302. By way of another example, the computing device 1100 can be or include the computing device 502. Pursuant to yet another example, the computing device 1100 can be or include the output device 704. The computing device 1100 includes at least one processor 1102 that executes instructions that are stored in a memory 1104. The instructions may be, for instance, instructions for implementing functionality described as being carried out by one or more components discussed above or instructions for implementing one or more of the methods described above. The processor 1102 may access the memory 1104 by way of a system bus 1106. In addition to storing executable instructions, the memory 1104 may also store health data, user health records, and so forth.

The computing device 1100 additionally includes a data store 1108 that is accessible by the processor 1102 by way of the system bus 1106. The data store 1108 may include executable instructions, health data, user health records, etc. The computing device 1100 also includes an input interface 1110 that allows external devices to communicate with the computing device 1100. For instance, the input interface 1110 may be used to receive instructions from an external computer device, from a user, etc. The computing device 1100 also includes an output interface 1112 that interfaces the computing device 1100 with one or more external devices. For example, the computing device 1100 may display text, images, etc. by way of the output interface 1112.

It is contemplated that the external devices that communicate with the computing device 1100 via the input interface 1110 and the output interface 1112 can be included in an environment that provides substantially any type of user interface with which a user can interact. Examples of user interface types include graphical user interfaces, natural user interfaces, and so forth. For instance, a graphical user interface may accept input from a user employing input device(s) such as a keyboard, mouse, remote control, or the like and provide output on an output device such as a display. Further, a natural user interface may enable a user to interact with the computing device 1100 in a manner free from constraints imposed by input device such as keyboards, mice, remote controls, and the like. Rather, a natural user interface can rely on speech recognition, touch and stylus recognition, gesture recognition both on screen and adjacent to the screen, air gestures, head and eye tracking, voice and speech, vision, touch, gestures, machine intelligence, and so forth.

Additionally, while illustrated as a single system, it is to be understood that the computing device 1100 may be a distributed system. Thus, for instance, several devices may be in communication by way of a network connection and may collectively perform tasks described as being performed by the computing device 1100.

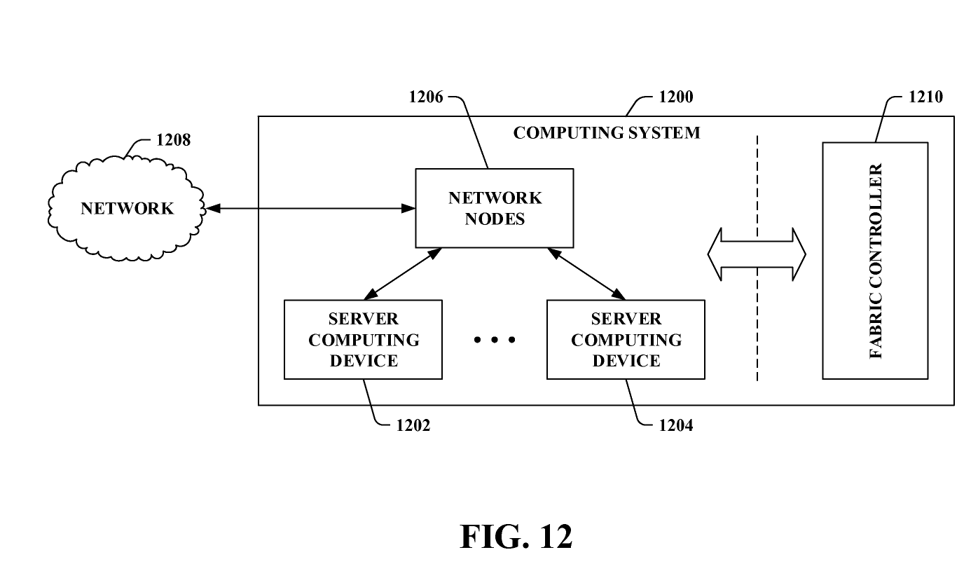

Turning to FIG. 12, a high-level illustration of an exemplary computing system 1200 that can be used in accordance with the systems and methodologies disclosed herein is illustrated. For instance, the computing system 1200 can be or include the computing system 102. Additionally or alternatively, the computing system 102 can be or include the computing system 1200.

The computing system 1200 includes a plurality of server computing devices, namely, a server computing device 1202, . . . , and a server computing device 1204 (collectively referred to as server computing devices 1202-1204). The server computing device 1202 includes at least one processor and a memory; the at least one processor executes instructions that are stored in the memory. The instructions may be, for instance, instructions for implementing functionality described as being carried out by one or more components discussed above or instructions for implementing one or more of the methods described above. Similar to the server computing device 1202, at least a subset of the server computing devices 1202-1204 other than the server computing device 1202 each respectively include at least one processor and a memory. Moreover, at least a subset of the server computing devices 1202-1204 include respective data stores.

Processor(s) of one or more of the server computing devices 1202-1204 can be or include the processor(s) 104. Further, a memory (or memories) of one or more of the server computing devices 1202-1204 can be or include the memory 106. Moreover, computer-readable storage of one or more of the server computing devices 1202-1204 can be or include the computer-readable storage 202.

The computing system 1200 further includes various network nodes 1206 that transport data between the server computing devices 1202-1204. Moreover, the network nodes 1202 transport data from the server computing devices 1202-1204 to external nodes (e.g., external to the computing system 1200) by way of a network 1208. The network nodes 1202 also transport data to the server computing devices 1202-1204 from the external nodes by way of the network 1208. The network 1208, for example, can be the Internet, a cellular network, or the like. The network nodes 1206 include switches, routers, load balancers, and so forth.

A fabric controller 1210 of the computing system 1200 manages hardware resources of the server computing devices 1202-1204 (e.g., processors, memories, data stores, etc. of the server computing devices 1202-1204). The fabric controller 1210 further manages the network nodes 1206. Moreover, the fabric controller 1210 manages creation, provisioning, de-provisioning, and supervising of virtual machines instantiated upon the server computing devices 1202-1204.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something.”

Various functions described herein can be implemented in hardware, software, or any combination thereof. If implemented in software, the functions can be stored on or transmitted over as one or more instructions or code on a computer-readable medium. Computer-readable media includes computer-readable storage media. A computer-readable storage media can be any available storage media that can be accessed by a computer. By way of example, and not limitation, such computer-readable storage media can comprise RAM, ROM, EEPROM, CD-ROM or other optical disk storage, magnetic disk storage or other magnetic storage devices, or any other medium that can be used to store desired program code in the form of instructions or data structures and that can be accessed by a computer. Disk and disc, as used herein, include compact disc (CD), laser disc, optical disc, digital versatile disc (DVD), floppy disk, and blu-ray disc (BD), where disks usually reproduce data magnetically and discs usually reproduce data optically with lasers. Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio and microwave are included in the definition of communication medium. Combinations of the above should also be included within the scope of computer-readable media.

Alternatively, or in addition, the functionality described herein can be performed, at least in part, by one or more hardware logic components. For example, and without limitation, illustrative types of hardware logic components that can be used include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the details description or the claims, such term is intended to be inclusive in a manner similar to the term “comprising” as “comprising” is interpreted when employed as a transitional word in a claim.

What is claimed is:

1. A method of controlling performance of a health assessment of a user in an entertainment venue, comprising:
accessing data in a health record of the user, the health record being retained in computer-readable storage, the user being located at the entertainment venue, and the entertainment venue comprising an attraction;
selecting a health parameter of the user to be measured as part of the health assessment performed in the entertainment venue, the health parameter being selected based on the data in the health record of the user;
controlling an interaction between the user and the attraction of the entertainment venue based on the health parameter to be measured; and
computing data indicative of the health parameter of the user based on a signal output by a sensor, the signal output by the sensor during the interaction between the user and the attraction of the entertainment venue.

2. The method of claim 1, controlling the interaction between the user and the attraction of the entertainment venue based on the health parameter to be measured further comprising transmitting a control signal to the attraction, the control signal configured to cause a physical modification of at least a portion of the attraction, the health parameter of the user being measurable based on a response of the user to the physical modification.

3. The method of claim 1, controlling the interaction between the user and the attraction of the entertainment venue based on the health parameter to be measured further comprising:
selecting at least a portion of a storyline based on the health parameter to be measured; and
causing the portion of the storyline to be presented to the user in the entertainment venue, the portion of the storyline configured to cause the user to interact with the attraction to enable the health parameter to be measured.

4. The method of claim 1, further comprising controlling the interaction between the user and the attraction of the entertainment venue based on the data in the health record of the user.

5. The method of claim 1, wherein a costume comprises the sensor, the costume being wearable for the attraction, the method further comprising receiving the signal output by the sensor of the costume worn by the user during the interaction between the user and the attraction.

6. The method of claim 1, wherein the attraction of the entertainment venue comprises the sensor.

7. The method of claim 1, further comprising storing, in the health record of the user:
the data indicative of the health parameter of the user; and
data indicative of a second health parameter of the user measured during an interaction between the user and a second attraction of the entertainment venue.

8. The method of claim 1, wherein a mobile device of the user comprises the computer-readable storage that retains the health record of the user.

9. The method of claim 1, wherein the attraction is a ride in the entertainment venue.

10. The method of claim 1, wherein the attraction is a group attraction, the user and a disparate user concurrently interact with the attraction while the signal is output by the sensor, the method further comprising:
selecting a health parameter of the disparate user to be measured;
concurrently controlling:
the interaction between the user and the attraction based on the health parameter of the user to be measured; and
an interaction between the disparate user and the attraction based on the health parameter of the disparate user to be measured; and
computing data indicative of the health parameter of the disparate user based on a disparate signal output by a disparate sensor, the disparate signal output by the disparate sensor during the interaction between the disparate user and the attraction of the entertainment venue.

11. The method of claim 1, further comprising:
detecting a health condition of the user based at least in part on the data indicative of the health parameter of the user;
generating treatment plan data for the user based on the health condition of the user; and
transmitting the treatment plan data to a mobile device of the user, wherein an application executable by the mobile device is personalized for the user based on the treatment plan data.

12. The method of claim 1, further comprising:
detecting a health condition of the user based at least in part on the data indicative of the health parameter of the user;
selecting educational data for the user based on the health condition of the user; and
presenting the educational data to the user.

13. The method of claim 1, wherein the interaction between the user and the attraction of the entertainment venue is controlled to perform a cardiac stress test on the user, the method further comprising:
transmitting a control signal to the attraction, the control signal configured to cause a physical modification of at least a portion of the attraction, the physical modification of the portion of the attraction configured to induce stress on the user; and
measuring data of the cardiac stress test before, during, and after induction of the stress on the user based on the signal output by the sensor, wherein the sensor comprises at least one of an electrocardiogram (ECG) device, a blood pressure measurement device, an ultrasound scanner, an x-ray device, or a magnetic resonance imaging (MRI) scanner.

14. The method of claim 1, wherein the interaction between the user and the attraction of the entertainment venue is controlled to perform a sleep test on the user, the method further comprising:
causing a portion of a storyline to be presented to the user in the entertainment venue, the portion of the storyline configured to prompt the user to sleep in the attraction; and
measuring data of the sleep test while the user sleeps in the attraction based on the signal output by the sensor, wherein the sensor comprises at least one of an electroencephalogram (EEG) device, an electrooculogram (EOG) device, an electromyogram (EMG) device, an electrocardiogram (ECG) device, a pressure transducer, a thermocouple, a pulse oximeter, or a microphone.

15. A computing system, comprising:
one or more processors; and
memory that comprises a health screening system that is executable by the one or more processors, the health screening system comprising:
a collection component that computes data indicative of a health parameter of a user based on a signal output by a sensor, the signal output by the sensor during an interaction between the user and an attraction of an entertainment venue, and the user being located at the entertainment venue;
a condition detection component that detects a health condition of the user based at least in part on the data indicative of the health parameter of the user;
a treatment component that generates treatment plan data for the user based on the health condition of the user; and
an output component that transmits the treatment plan data to a mobile device of the user, wherein an application executable by the mobile device is personalized for the user based on the treatment plan data.

16. The computing system of claim 15, the health screening system further comprising:
an interface component that accesses data in a health record of the user, the health record being retained in computer-readable storage;
an analysis component that selects a health parameter of the user to be measured as part of a health assessment performed in the entertainment venue, the health parameter being selected based on the data in the health record of the user; and
a control component that controls the interaction between the user and the attraction of the entertainment venue based on the health parameter to be measured.

17. The computing system of claim 15, the health screening system further comprising a storyline management component that causes a storyline to be presented to the user during the interaction between the user and the attraction of the entertainment venue, wherein the application continues to cause the storyline to be presented to the user.

18. The computing system of claim 15, wherein the treatment plan data causes the application to output a health suggestion via the mobile device.

19. A computing system, comprising:
one or more processors; and
memory that comprises a health screening system that is executable by the one or more processors, the health screening system comprising:
a control component that:
transmits a first control signal to a first attraction in an entertainment venue, the first control signal configured to cause a first physical modification of at least a portion of the first attraction, a first health parameter of a user being measurable based on a response of the user to the first physical modification; and
transmits a second control signal to a second attraction in the entertainment venue, the second control signal configured to cause a second physical modification of at least a portion of the second attraction, a second health parameter of the user being measurable based on a response of the user to the second physical modification; and
a collection component that:
computes data indicative of the first health parameter of the user based on a first signal output by a first sensor, the first signal output by the first sensor during an interaction between the user and the first attraction; and
computes data indicative of the second health parameter of the user based on a second signal output by a second sensor, the second signal output by the second sensor during an interaction between the user and the second attraction of the entertainment venue.

20. The computing system of claim 19, further comprising:
an analysis component that selects the first health parameter of the user and the second health parameter of the user to be measured as part of a health assessment performed in the entertainment venue;
wherein the control component transmits the first control signal and the second control signal based on the first health parameter of the user and the second health parameter of the user selected to be measured.
